(19) United States
(12) Patent Application Publication
(10) Pub. No.: US 2007/0270918 A1
(43) Pub. Date: Nov. 22, 2007

(54) Appliance and Method for Measuring an Emg Signal

(75) Inventors: Maxime De Bel, Waterloo (BE); Francis Cantraine, Bruxelles (BE); Pierre Mathys, Braine Le Chateau (BE)

Correspondence Address: REINHART BOERNER VAN DEUREN P.C., 2215 PERRYGREEN WAY, ROCKFORD, IL, 61107, US

(73) Assignee: Universite Libre De Bruxelles, Brussels (BE)

(21) Appl. No.: 11/667,439

(22) PCT Filed: Nov. 10, 2005
(86) PCT No.: PCT/BE05/00162

§ 371 Date: May 9, 2007

(30) Foreign Application Priority Data
Nov. 10, 2004 (EP) 04447248.8

(51) Int. Cl. A61N 1/02

(52) U.S. Cl. 607/48

(57) ABSTRACT

The present invention relates to an appliance for the direct measurement, display, processing and transmission, remotely, of electromyographic signals (EMG) comprising: an electrical stimulator (1) comprising electrodes for the excitation of a peripheral motor nerve; a pair of electrodes (31), for the acquisition of the EMG response at the level of the muscle associated with this peripheral nerve; an acquisition chain (2) driven by a micro-controller (3, 39), presenting means of conditioning of the input signal, comprising at least one differential preamplifier (22, 33), a bandpass filter (25, 34) and an analog/digital converter (ADC) (26, 310), said acquisition chain (2) being linked, via a standardized interface (5), to a computer (4) comprising means of storage (9) and of display of the EMG signals acquired as well as an executable program for effecting the interface with the user (6) and utilizing the data stored; characterized in that the acquisition chain (2) comprises means of automatic adjustment of the amplification gain of the EMG signal (23, 24, 311, 38), via the microcontroller, in such a way that the EMG signal covers the largest possible part of the input voltage span of the ADC (26, 310), hence with conservation of resolution, when the amplitude of the EMG signal decreases.

### SUBJECT OF THE INVENTION

The present invention relates to a novel appliance and to a novel method of measuring electromyograms.

When a muscle is under activity, it is possible to gather a low amplitude (bio)electrical signal by placing electrodes on it. All these signals, taking the form of electrical potentials, are called an electro-myogram (EMG).

The aim of an EMG analysis is to obtain information on the state and the functioning of the muscles through quantification of the electromuscular activity. This measurement is performed by means of electrodes applied on or under the skin. A signal is detected, manifesting the activity of the subjacent muscle.

### BACKGROUND AND PRIOR ART

Electro-stimulation is known and consists in exciting a peripheral motor nerve by means of electrical pulses so as to cause, in an external manner, hence without the intermediary of the brain, the reaction of the muscle associated therewith.

Two major fields of application are based on this technique for proposing solutions associating electro-stimulation with measurement of the EMG.

The first field of application is concerned with general anesthesia and, in particular, with the monitoring of this anesthesia. In this regard, the patient is injected various drugs aiming:
to ensure amnesia and sleep through unconsciousness;
to anaesthetize to pain through analgesics; and
to allow muscle relaxation.

This latter task is ensured by curare which decreases the number of active muscle fibers; in this case, one resorts to EMGs to evaluate the rate of muscle relaxation.

This evaluation of the muscle relaxation is confronted with a certain number of difficulties of measurement:
a decrease in the amplitude of the EMGs during curarization;
a noisy environment, in particular through electromagnetic pollution;
the requirement that the initialization phase, that is to say the time for setting up the electrodes and calibrating the appliance, relatively short.

A large number of documents propose using the measurement of the EMG for anesthesia monitoring applications of the measurement of the EMG, possibly associated with a measurement of the EEG, which usually work according to the “stimulation-response” scheme. As an example, the following documents may be cited: U.S. Pat. No. 4,291,705, GB-A-2 113 846, U.S. Pat. No. 4,595,018, KR-A-9 004 899, U.S. Pat. No. 5,300,096, U.S. Pat. No. 4,291,705, U.S. Pat. No. 6,224,549, WO-A-02 053012, WO-A-99 41682. These documents have been described in details in the priority application and are integrated by reference into the present application.

In most cases, the measurements are marred by a stimulation artifact. Moreover, for all these appliances or methods, a loss of signal is obtained when the EMGs decrease in amplitude.

More particularly, document U.S. Pat. No. 6,083,156 published on Jul. 4, 2000 describes an integrated, portable and autonomous appliance comprising:
an electrical stimulator;
a pair of electrodes;
an acquisition chain (amplifier, bandpass filter, an ADC, etc.);
and driven by a portable computer.

Likewise, the document “A gated differential amplifier for recording physiological responses to electric stimulation” describes an amplifier comprising means of attenuation of the stimulation artifact by changing the gain before and after stimulation:
unit gain during stimulation;
gain of 1000 (in the 300 Hz-25 kHz band) after stimulation.

In these documents, the change of gain is used to minimize the effects of the artifact and not to keep the resolution constant despite the EMGs varying in amplitude.

It is therefore appropriate to differentiate the EMG signals, resulting from electrical stimulation, from the spontaneous EMG signals, resulting from a voluntary movement of the muscle.

Another major field of application is the use of EMGs to embody an appliance which is suitable for kinesitherapeutic applications. Specifically, in this case:
no muscle relaxants are used and therefore the electrical signals are much larger;
spontaneous potentials are measured therein, the measurements are not disrupted by the stimulation artifact.

In document U.S. Pat. No. 5,300,096 “ELECTROMYOGRAPHIC TREATMENT DEVICE”, one wishes to print, following a stimulation performed by means of an electrical pulse, a muscular response or reaction that will be measured so as to adapt the stimulation to the required results.

Documents U.S. Pat. No. 5,300,096 and WO-A-2005/046787 describe an appliance which uses an electrical muscle stimulator which converts the EMG signals into digital signals allowing the analysis and display with a computer program which makes it possible to assist the therapist graphically in the execution of kinesitherapy exercises.

### AIMS OF THE INVENTION

The purpose of the present invention is to propose a solution which makes it possible to be freed from the drawbacks of the prior art.

According to a first object, the invention is aims to provide an appliance for measuring electro-physiological signals of EMG type associated with an electro-stimulation, and which is preferably portable, autonomous, very compact, reliable, flexible, easy to use, in conformity with the electrical safety standards (limitation of the default current) and inexpensive to manufacture.

A first important aim of the invention is to provide an appliance which can be suitable for anesthetic applications and/or kinesitherapeutic applications which can perform reliable measurements despite the decrease in amplitude of the EMG signals.

Subsidiarily, a complementary aim of the present invention is to allow easy and automatic control of the correct placement of the measurement and stimulation electrodes.

A second important aim of the present invention is to allow fast calibration of the appliance, especially for anesthesia applications, taking into account a possible stimulation artifact, while having an accurate determination of the amplitude of the supra-maximal excitation.

A complementary aim of the invention is to provide an appliance which can be linked or driven by a (network of) remote computer(s), possibly by means of a wireless connection.

According to a second object, the present invention is directed towards providing a method of measuring electrophysiological signals of EMG type associated with an electro-stimulation.

A final object of the present invention is directed towards proposing the use of the appliance or of the method which are described above for therapeutic and diagnostic applications.

### PRINCIPAL CHARACTERISTIC ELEMENTS OF THE INVENTION

A first object of the present invention relates to an integrated and autonomous appliance for the direct measurement, display, remotely processing and transmission of electromyographic signals (EMG) described according to the terms of claim 1, and which therefore comprises:
an electrical stimulator comprising electrodes for the excitation of a peripheral motor nerve;
a pair of electrodes, for the acquisition of the EMG response at the level of the muscle associated with this peripheral nerve;
an acquisition chain driven by a microcontroller, exhibiting means of conditioning the input signal, comprising at least one differential preamplifier, a bandpass filter and an analog/digital converter (ADC), said acquisition chain being linked, via a standardized interface, to a computer comprising means of storage and display of the EMG signals obtained as well as an executable program for effecting the interface with the user and utilizing the data stored.

The innovation resides in the automatic adaptation of the amplification gain of the EMG signal measured as to optimize the use of the resolution of the analog/digital converter of the system. Stated otherwise, the invention makes it possible to provide a solution for automatic gain control with a maximum accuracy (that is to say a minimum relative quantization error).

A first area of application is directed towards proposing the use of the appliance according to the present invention in the field of anesthesia in which the evaluation of the rate of muscle relaxation during curarization is measured. In this case, this involves measuring the response following electro-stimulation. It is therefore the “stimulation-response” mode.

Through the use of the appliance according to the present invention for this type of application, it is observed that the resolution is maintained, even when the amplitude of the EMG signal decreases over time. Moreover, the invention helps to solve the problem of the signal-to-noise ratio decrease related to the decrease in the amplitude of the EMG signal during curarization. More generally, the invention allows effective measurement in a noisy environment (electromagnetic pollution).

A second area of application is directed towards proposing the use of the appliance in the so-called “inverted” mode for kinesitherapy applications. According to this mode, the measurement chain periodically samples the monitored muscles and triggers an electro-stimulation when the EMG related to a voluntary contraction exceeds a programmable threshold. This makes it possible to improve muscular rehabilitation by assisting the re-education movements.

According to this mode of use of the appliance intended for kinesitherapy applications, it is observed that:
the EMG is used as a measurement tool. The objective is in particular to analyze the patterns (models) of muscular recruitment in standardized exercises so as to highlight anomalies;
the electro-stimulation, on the other hand, is used as treatment tool. The motor nerve of the muscle to be rehabilitated is subjected to trains of electric pulses so as to cause well-determined contraction sequences. The current user is however limited to predefined trains of rectangular pulses and this technique lacks of flexibility.

For both kinesitherapy and anaesthesia applications, the present invention aims to propose a solution which allows automatic adjustment of the gain of the amplifiers so as to extend the EMG over the totality of the input voltage span of the analog/digital converter and this even when the EMG varies in amplitude.

Another important aim of the present invention is, in the particular case of anesthesia applications, to solve the problem of the stimulation artifact. Indeed, when the EMG decreases in amplitude (on account of the effects of the curare), a moment occurs when the amplitude of the artifact becomes greater than the amplitude of the EMG. In order to be able to continue to amplify the EMG with the optimum gain without any risk of saturation, following excessive amplification of the artifact, the appliance short-circuits the measurement electrodes for the duration of the artifact.

In this case, the automatic adjustment of the gain and the short-circuiting of the electrodes are therefore two distinct mechanisms which, when associated, make it possible to keep the relative quantization error constant regardless of the amplitude of the stimulation artifact.

This aim is achieved by the solutions proposed in the subsidiary claims 2 to 5.

An aim complementary to the previous ones is directed towards solving the problems related to the offset, which appear when working in “stimulation-response” mode with short-circuiting of the measurement electrodes. Indeed, when the DC component at the output of the preamplifier is not zero, the short-circuiting of the measurement electrodes causes perturbations at the output of the bandpass filter.

Several solutions have been envisaged and are described in details in claims 6 to 12. In particular:
a solution which considers the elimination of this problem by programming;
a solution which proposes a hardware compensation in which the removal of the high-pass filter and the addition of an extra programmable circuit at the output of the preamplifier are provided;
another solution which proposes a hardware compensation and in which the removal of the high-pass filter and the replacing of the instrumentation amplifier by an amplifier with compensation external resistor for the offset are provided.

Preferred embodiment of the invention are detailed in the dependent claims 13 to 21.

A second object of the present invention is described in claim 22 which relates to a method for automatically adjusting the gain applied to the input signal and maintaining the maximum resolution of the analog/digital converter in the above-mentioned measurement appliance, depending on whether this appliance is used in “stimulation-response” mode for applications of monitoring muscle relaxation during curarization or whether it is used in so-called “inverted” mode for applications, for example in kinesitherapy.

Again, proposals of solutions for solving the problems mentioned hereinabove are described in dependent claims 22 to 27 for a method.

Finally, therapeutic applications are alluded to and described in claims 28 to 30.

### BRIEF DESCRIPTION OF THE FIGURES

FIG. 1 represents the block diagram of the stimulation and measurement acquisition system according to the invention.

FIG. 2A represents a triangular signal shape.

FIG. 2B represents the parameterization of a stimulation sequence.

FIG. 2C represents a trapezoidal signal shape.

FIG. 3 represents the diagram of the acquisition chain principle according to the invention.

FIG. 4 represents diagrammatically the method of automatic adjustment of the gain.

FIG. 5 represents an EMG signal with stimulation artifact such that V_{EMG}>V_{ARTIFACT}.

FIG. 6 represents an EMG signal with stimulation artifact such that V_{EMG}<V_{ARTIFACT}.

FIG. 7 shows the saturation of the amplifier following overly large amplification of the stimulation artifact.

FIG. 8 shows the adjustment of the gain for an EMG with stimulation artifact such that V_{ARTIFACT}>V_{EMG}.

FIG. 9 shows the diagram of the acquisition chain.

FIG. 10A represents diagrammatically the short-circuiting of the measurement electrodes.

FIG. 10B shows the sequence of times during which the operations are performed during a short-circuiting of the measurement electrodes.

FIG. 11 represents a diagram of the measurement chain in a preferred embodiment of the appliance according to the present invention which makes it possible to solve the problems related to the short-circuiting of the offset.

FIGS. 12A and 12B represent a patient trial using a software compensation to solve the problem of the offset.

FIG. 13 represents a diagram of the measurement chain for a preferred embodiment of the present invention which proposes a hardware compensation for solving, according to a first embodiment, the problem related to the offset.

FIGS. 14A and 14B represent a patient trial which envisage a combination of the software compensation as performed and represented in FIGS. 13A and 13B associated with a hardware compensation as described in FIG. 13.

FIG. 15 represents a diagram of the measurement chain according to another embodiment which allows an alternative hardware compensation to the problems related to the short-circuiting of the offset.

FIG. 16 corresponds to the signal of FIG. 10B according to whether the system is operating in “stimulation-response with short-circuiting of the electrodes” mode (mode 1) or in “stimulation-response without short-circuiting of the electrodes” mode (mode 2).

FIG. 17 diagrammatically represents the network arrangement for a polytopic measurement.

FIG. 18 diagrammatically represents a closed-loop acquisition, in an operating theatre.

FIG. 19 graphically represents a search of intensity leading to a “supra-maximal” excitation.

FIG. 20 represents the amplitude of the EMG signal as a function of the intensity of the electrical pulses.

### DESCRIPTION OF A PREFERRED EMBODIMENT OF THE INVENTION

1. Presentation of the Appliance

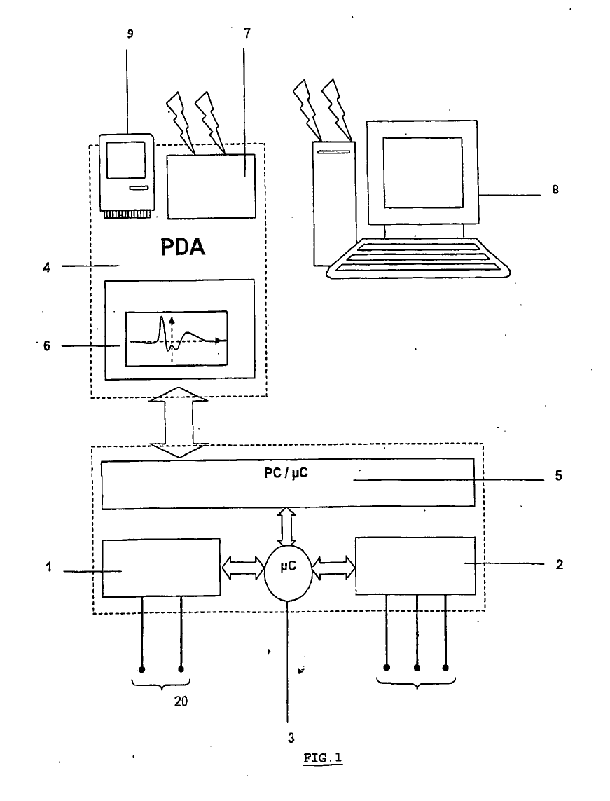

The appliance according to the invention, illustrated diagrammatically in FIG. 1, comprises a current source 1 linked to stimulation electrodes (20) allowing the excitation of a peripheral motor nerve and an acquisition chain 2 specially adapted to the measurement of the electromyographic potentials, spontaneous and/or evoked by way of measurement electrodes 31.

At the heart of the system is found a microcontroller 3 which has to ensure the driving and the synchronization of the various modules of the system in real time as well as the communication with the computer 4 via a standardized interface 5 (RS-232, USB, RS-485, etc.).

The EMG signal and/or its parameters may be viewed on the display screen 6.

Although the system may be driven from any workstation comprising a standard communication port (RS-232, USB, RS-485, etc.), it is advisable to preferably use a PDA (Personal Digital Assistant) 4 to effect the user interface so as to ensure portability, autonomy and safety of the whole system (see hereinbelow, section 1.7).

In order to facilitate its integration into a medical monitoring system, the system is capable of establishing a wireless communication with a central computer 8 via a WIRELESS transmitter/receiver 7 integrated into the PDA.

The EMG responses may be recorded in the non-volatile memory 9 available on the PDA (SD, CompactFlash, etc.).

Of course, a program makes it possible to utilize the data originating from the onboard system while serving as user interface.

The appliance can operate in four different modes:
Mode 1 “Stimulation-response”: for anaesthetic applications. In this mode, the appliance stimulates a peripheral motor nerve by means of electrical pulses and thereafter processes the EMG evoked resulting from the stimulation;
   Mode 2 “Inverted mode”: for kinesitherapy applications. In this mode, the measurement chain periodically samples the muscle monitored and triggers an electro-stimulation when the EMG related to a voluntary contraction exceeds a programmable threshold. This makes it possible to improve muscle rehabilitation by assisting the re-education movements.
   Mode 3 “acquisition only”: mode not described.
   Mode 4 “stimulation only”: mode not described.

Depending on the application sought, the appliance may present one or more modes of operation. The user may therefore choose
the type of stimulation to be delivered;
the mode of display of the response curve; etc.

The subsequent description is devoted to one or more embodiment(s) of the present invention which highlights (highlight) the advantages which result from the grouping together of the stimulator and the EMG acquisition chain in the same appliance.

1.1 User Interface

The interface should, preferably:
comprise a color screen of reasonable size for clear display of the EMG and touch-sensitive for easy interaction with the user;
be easily adaptable to the application (ergonomics and signal processing);
offer some customization (pruning of menus, pre-configurations adapted to the user).

Moreover, to facilitate the prototyping and the development of the interface, the storage of data and the post-processing are done on a pocket computer with touch-sensitive screen. Another solution may also be considered wherein the PDA is replaced with an equivalent onboard system integrated into the appliance, thereby enhancing portability.

Choice of the Operation Mode of the Program

Recording:

In this mode, the system performs the acquisition of the EMG, saves the response on the non-volatile memory, displays the curve, performs a processing of the curve and displays the determining parameters.
Reading:

In this mode, the user reads the EMGs recorded. The system performs a reading in the memory of the appliance, displays the signal, performs a processing of the curve and displays the determining parameters.

Evaluation of the Critical Parameters

The evaluation of the muscle relaxation may be done in particular by measuring the ratio of the peak-to-peak amplitude of the curarized EMG to the peak-to-peak amplitude of a reference EMG (for example T₁/T₀, T₄/T₁, etc.) or by measuring the ratio of the areas (for example S₁/S₀, S₄/S₁, etc.) of the rectified EMGs (that is to say the potentials taken as absolute value). Nevertheless, many parameters may be used. The software makes it possible to conduct the analysis completely, both in real time and by post-processing.

1.2 The Stimulator

Delivered Signals

The system comprises a stimulator that can preferably work in two different modes. In the first mode, the stimulator delivers stimulation sequences programmable by the user. In the second, it delivers the pulse trains customarily used in anaesthesia.

Programmed Mode

The electro-stimulator contains a series of sequences of rectangular pulses prerecorded in its memory, such as ST (Single Twitch), TOF (Train Of Four), TS (Tetanic Stimulation) or DBS (Double Burst Stimulation).

The intensity, the width of the pulses and the period between two successive sequences have a default value but are reparametrizable by the user within the range:
amplitude of the pulses: 0-150 mA in 5 kΩ;
width of the pulses: 0-1000 μs;
period separating two successive sequences: 10-60 s.
   Programmable Mode

The user can choose at one and the same time the shape of the signal (trapezoidal, sinusoidal, triangular, rectangular, of arbitrary shape), its frequency and its amplitude.

Rectangular:

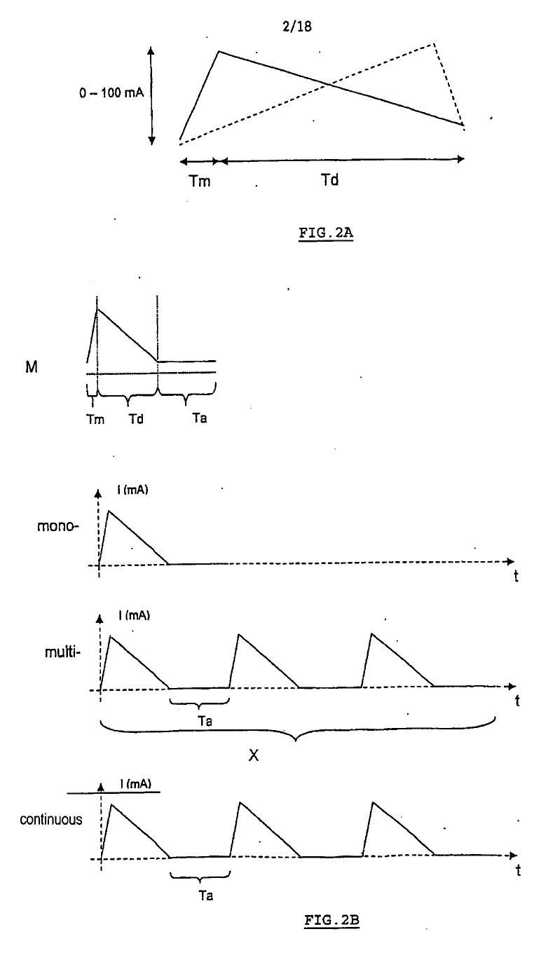

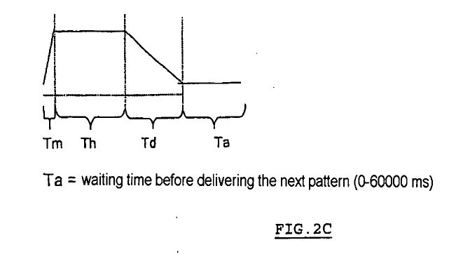

amplitude: 0-150 mA in 5 kΩ;
width of the pulses: 0-1000 μs.
   Trapezoidal (see FIG. 2C):
amplitude: 0-150 mA in 5 kΩ;
rise time (Tm): 15 μs-5 ms;
top time (Th): 0-1000 μs;
fall time (Td): 15 μs-5 ms.
   Triangular (see FIG. 2A):
amplitude: 0-150 mA in 5 kΩ;
rise time (Tm): 15 μs-5 ms;
fall time (Td): 15 μs-5 ms.
   Sinusoidal:
amplitude: 0-150 mA;
frequency: 0-200 Hz.

Ta is the waiting time before delivering the next pattern. Once the pattern (M) has been established, the user can choose to work in:
mono-pulse mode: to deliver a single pattern;
multi-pulse mode: to deliver a determined number of patterns, fixing the time Ta between two consecutive patterns;
continuous mode: to electro-stimulate continuously, fixing the time Ta which separates two consecutive patterns.

FIG. 2B illustrates the parametrization of a stimulation sequence.

Safety Aspects

The stimulator performs regularly or on demand the measurement of impedance between the stimulation electrodes according to medical standards.

1.3 The Acquisition Chain

Principle Diagram

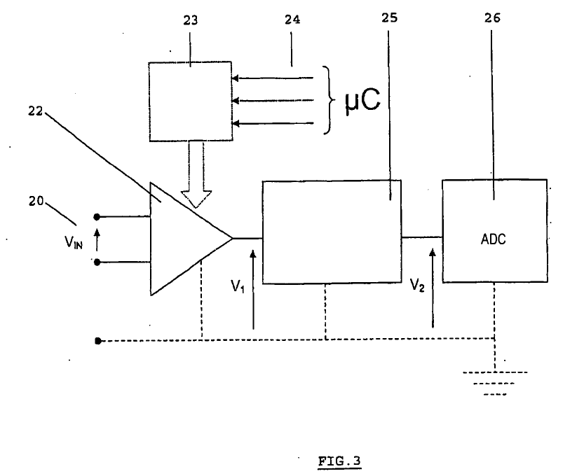

The acquisition chain 2, represented diagrammatically in FIG. 3, is intended to amplify the signal originating from the measurement electrodes 20 with the aid of a differential amplifier 22 so as to obtain a signal “V₁”, to filter “V1”, through a filter 25 and to extract therefrom the undesirable frequencies and to perform the analog/digital conversion 26 of the signal “V₂” thus conditioned.

To ensure maximum resolution in the analog/digital conversion 26, the system for automatic adjustment of the gain 23 controlled 24 from the microcontroller makes it possible to amplify the input signal 20 so as to make best use of the voltage span of the converter.

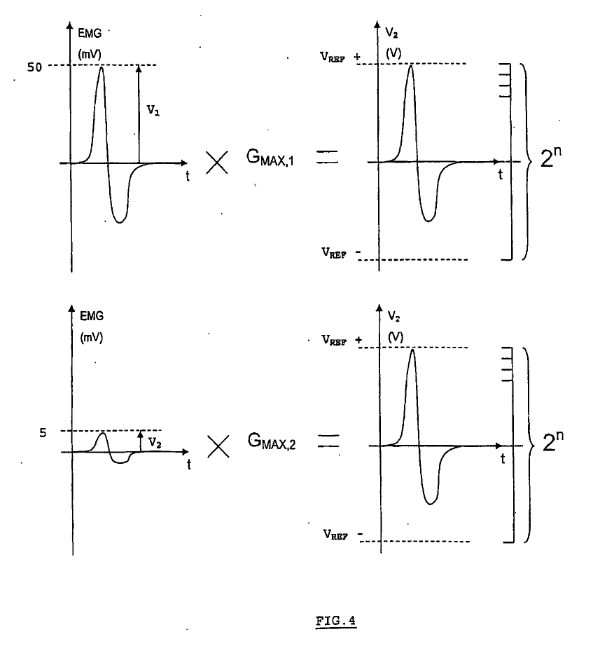

By choosing as gain (see FIG. 4):

$${G}_{MAX} = \frac{{V}_{REF}}{{V}_{MAX}} , \qquad ( 1 )$$

where:
V_{REF} is the half of the input span of the analog digital converter and
V_{MAX} is the peak value of the signal to be measured,
the amplitude of the signal “V₂” at the input of the converter will use the “totality” of its voltage span. Still according to FIG. 4, we have:

$${G}_{MAX , 1} = \frac{{V}_{REF}}{{V}_{1}} , \\ {G}_{MAX , 2} = \frac{{V}_{REF}}{{V}_{2}} ,$$

with
V_{REF}=max(|V_{REF+}|, |V_{REF -}|).
Problem Related to the Stimulation Artifact

The stimulation causes an artifact which disturbs the EMG measurement of low amplitude.

The EMG signals being of relatively low amplitude and collected in a fairly noisy environment, they should be amplify to the maximum and as near as possible to the measurement site.

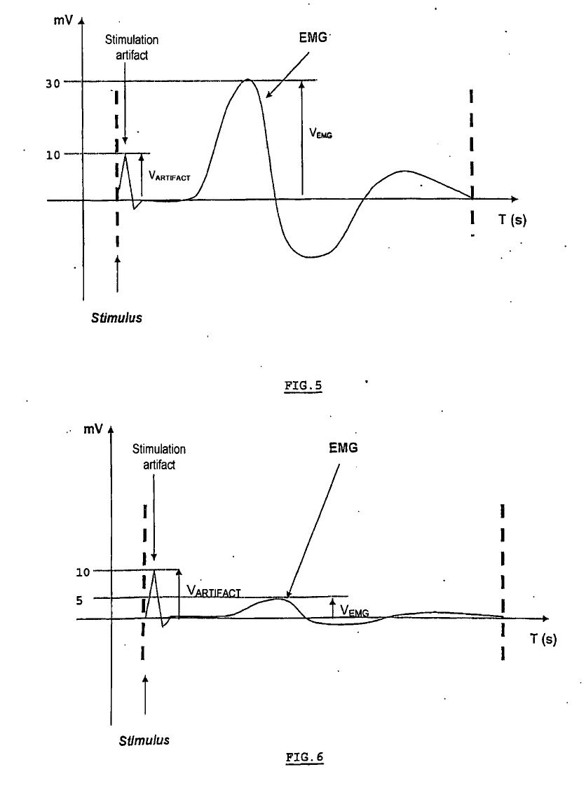

FIG. 5 shows that, for EMGs of suitable amplitude (when the patient is not curarized), the amplitude of the muscle response is generally higher than the amplitude of the stimulation artifact.

Condition (1) and V_{MAX}=V_{EMG} imply that

$${G}_{MAX} = \frac{{V}_{REF}}{{V}_{EMG}} . \qquad ( 2 )$$

The maximum gain of the amplifier is therefore inversely proportional to the amplitude of the EMG signal.

As the increasing in the concentration of curare causes a progressive and considerable decrease in the amplitude of the EMGs, a moment occurs at which the amplitude of the EMG becomes smaller than the one of the stimulation artifact (see FIG. 6).

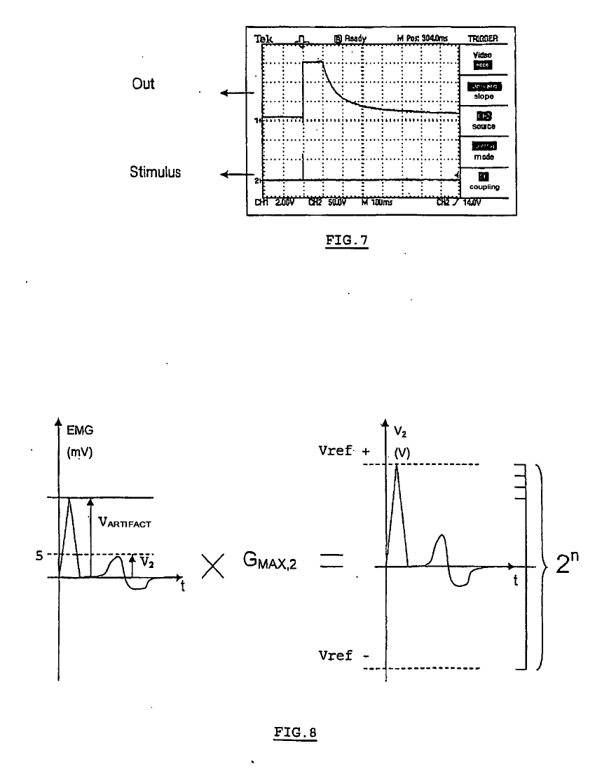

The choice of the gain then arises. A similar dimensioning to the one of the expression (2) based on the amplitude of the EMG could cause a saturation of the amplifier following excessive amplification of the stimulation artifact, as shown in FIG. 7.

The maximum gain of the amplifier therefore no longer depends on the amplitude of the EMG signal but indeed on the amplitude of the stimulation artifact, thereby engendering a poor signal-to-noise ratio (SNR) for small EMGs.

FIG. 8 shows the adjustment of the gain for an EMG with stimulation artifact such that V_{ARTIFACT}>V_{EMG}.

If V_{ARTIFACT}>V_{EMG}, then

$${G}_{MAX} = \frac{{V}_{REF}}{{V}_{ARTIFACT}} , \qquad ( 3 )$$

and if V_{EMG}>V_{ARTIFACT}, then

$${G}_{MAX} = \frac{{V}_{REF}}{{V}_{EMG}} , \qquad ( 4 )$$

where
V_{EMG} is the peak amplitude of the EMG,
V_{ARTIFACT} is the peak amplitude of the artifact,
V_{REF} is the half of the input span of the ADC.

Two methods are used, depending on the operation mode, to minimize the influence of the artifact on the measured signal and which make it possible to usefully exploit the whole of the input span of the ADC.

Solutions Implemented

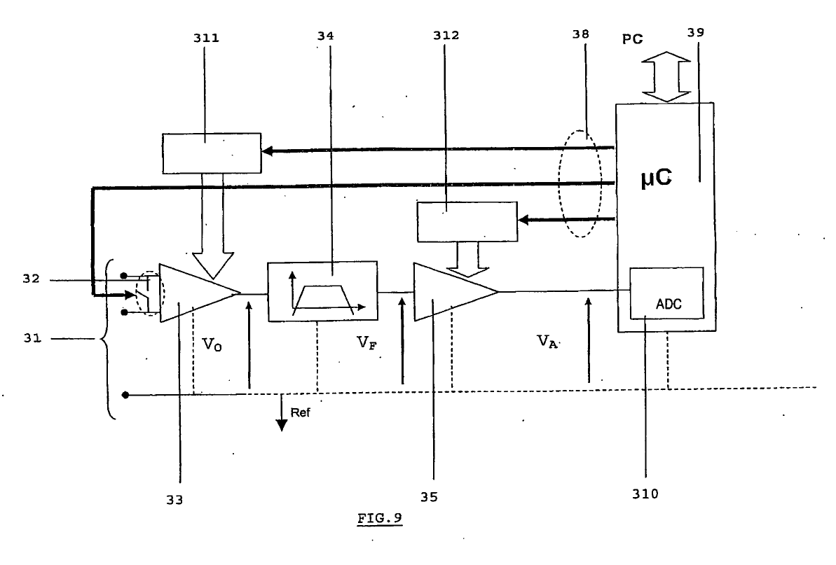

On the left of the block diagram represented in FIG. 9 may be distinguished the connector 31 to the measurement electrodes and the reference electrode. The system performs a differential preamplification 33 in order to minimize the common-mode noise picked up by the human body.

Between the preamplifier 33 and the connector for the electrodes 31 may be seen the presence of a relay 32, driven by an output 38 of the microcontroller 39, allowing the short-circuiting of the measurement electrodes.

The preamplified signal “V₀” passes through a bandpass filter 34 so as to preserve only its useful frequencies (10-1000 Hz).

The filtered signal “V_{F}” may possibly be reamplified 35 so as to best lie within the input voltage span of the analog digital converter 310 (see “mode 2” hereinbelow).

Two distinct modules 311 and 312 make it possible to independently adjust the gain of the preamplifier 33 and the one of the amplifier 35 via certain output lugs 38 of the microcontroller 39.

a) Stimulation Response with Short-Circuiting of the Measurement Electrodes Mode (Mode 1)

In this case, a masking of the signal is carried out. This method consists in short-circuiting the acquisition electrodes for the duration of the stimulation artifact. It requires the stimulator to be coupled to the EMG acquisition chain and that it provides a synchronization signal.

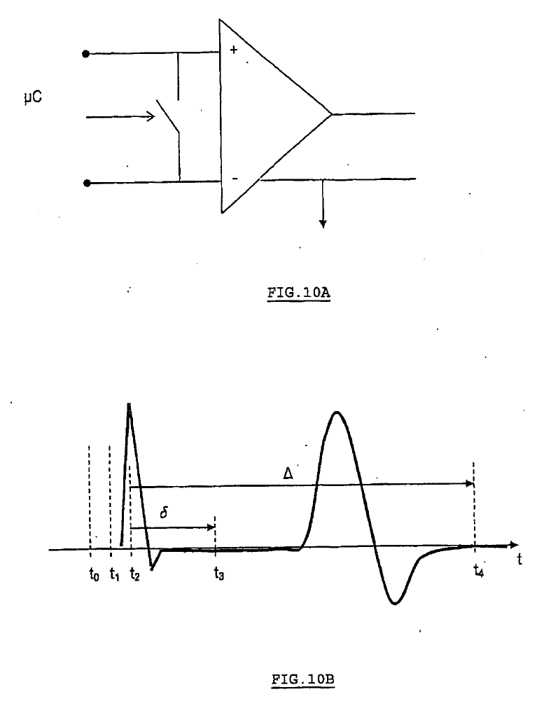

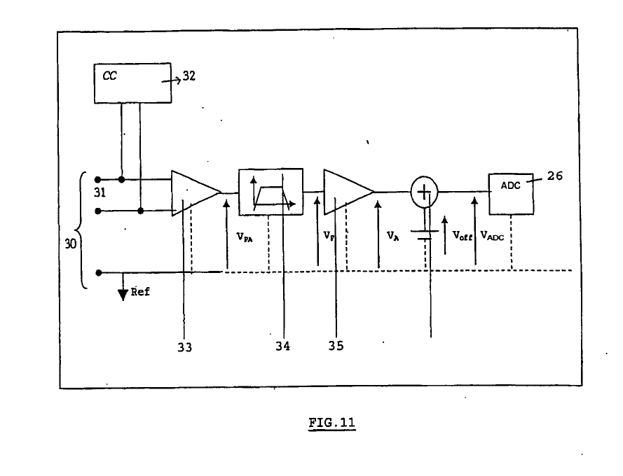

FIG. 10A shows the principle of the short-circuiting of the measurement electrodes by the microcontroller μC. FIG. 10B shows the time sequence at which the operations are performed during a short-circuiting of the measurement electrodes, where:
t₀ is the instant of short-circuiting of the measurement electrodes;
t₁ is the instant at which stimulation starts;
t₂ is the instant at which stimulation ends and acquisition starts;
t₃ is the instant at which the measurement electrodes are short-circuited;
t₄ is the instant at which acquisition ends;
δ is the duration of short-circuiting of the electrodes after stimulation and
Δ is the recording period.

In this way, the stimulation artifact has totally disappeared from the measured signal and the condition V_{EMG}>V_{ARTIFACT} is constantly satisfied.

The gain of the preamplifier may be dimensioned immediately in an optimal manner. The time between the end of the stimulation and the opening of the relay short-circuiting the electrodes is made programmable for the user.

Solution of Problems Related to the Short-Circuiting of the Offset

The problems related to the offset appear when working in “stimulation-response” mode with short-circuiting of the measurement electrodes, that is to say in applications directed towards anaesthesia.

The presence of the offset is related to the contact potentials at the level of the electrode/gel and gel/skin interfaces. If these potentials were equal, they would compensate one another at the level of the preamplifier and would not disrupt the measurement chain. Their asymmetry gives rise to a DC component at the output of the preamplifier. This asymmetry may be significantly reduced by good preparation of the skin.

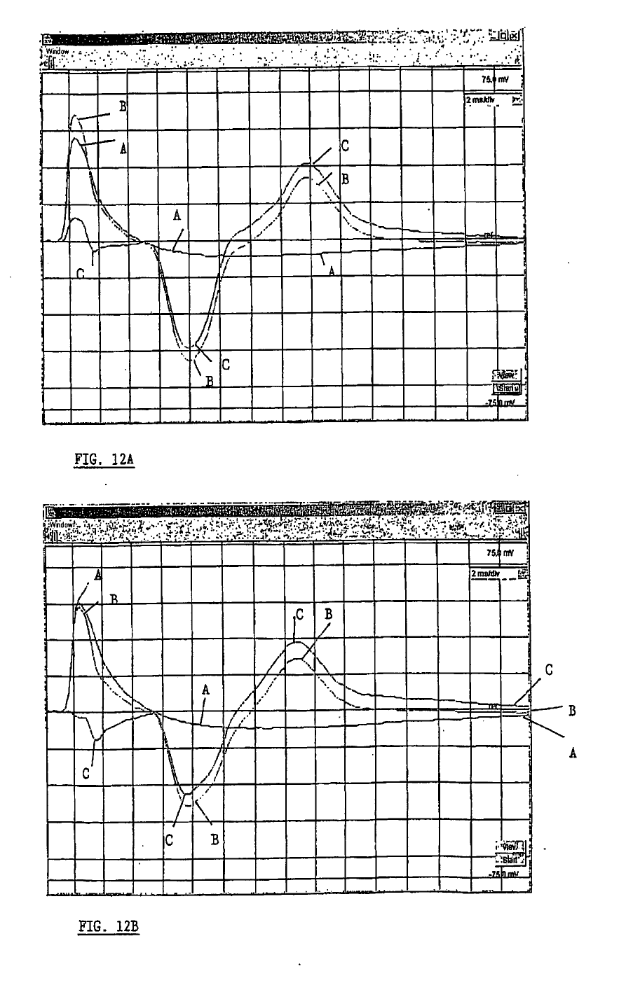

FIG. 11 represents a simplified diagram of the measurement chain, in which the measurement electrodes 31 are short-circuited by a short-circuiting element 32.

The short-circuiting of the DC component causes perturbations at the output of the bandpass filter.

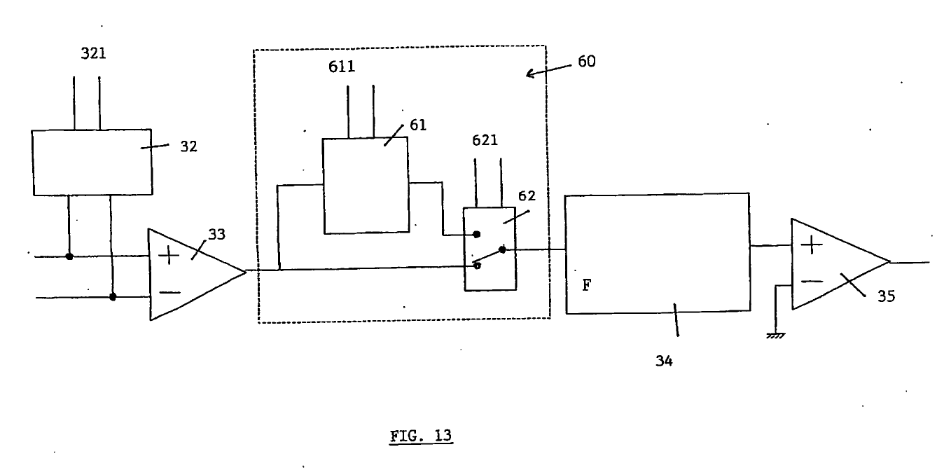

A first form of execution makes it possible to propose a solution to this problem related to the offset by considering a software compensation. This method essentially comprises three steps based on the principle of superposition as illustrated in FIG. 12. They are described hereinbelow:
short-circuiting the measurement electrodes without electro-stimulating and recording the perturbation at the output of the measurement chain (Curve A);
performing the measurement of the EMG evoked; the signal measured is then the superposition of the EMG evoked and of the perturbation related to the high-pass filter (Curve B);
subtracting the perturbation signal from the measured signal and displaying the result (Curve C).

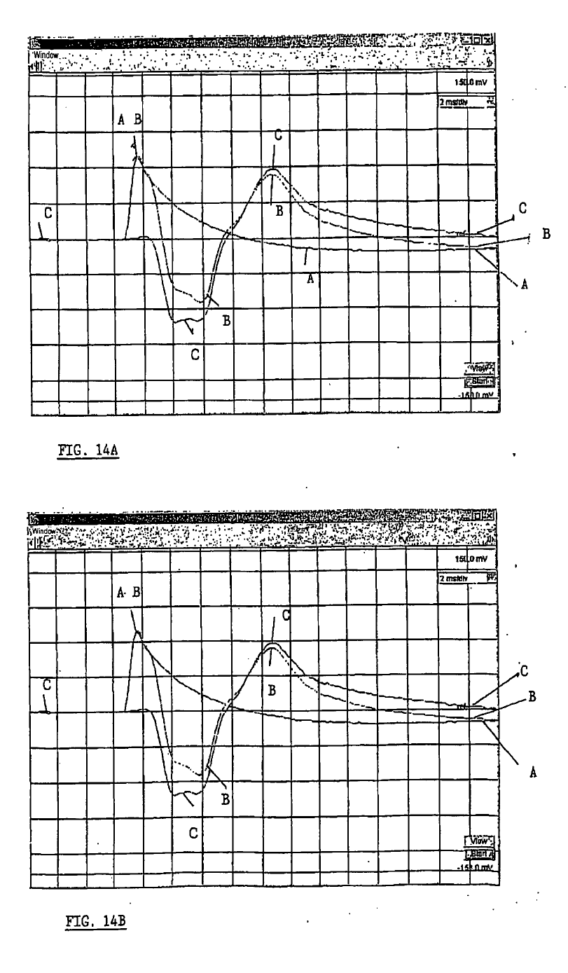

A second form of execution makes it possible to propose a solution to the problem related to the offset termed “hardware compensation”. This hardware compensation aims to prevent abrupt variations of the input voltage of the high-pass filter by storing the value of the offset, before the short-circuiting of the measurement electrodes and by keeping this voltage at the input of the filter throughout the duration of the short-circuit. The hardware compensation illustrated in FIG. 13 is defined by the following steps:
before stimulating, the output of the preamplifier 33 is sampled and the sample is stored by means of a Sample & Hold sampler 61;
the output of the sampler 61 is connected to the input of the bandpass filter 34 by means of an analog multiplexer 62;
the measurement electrodes are short-circuited by the element 32;
the measurement electrodes are de-short-circuited;
the input of the bandpass filter 34 is reconnected to the output of the preamplifier.

According to another embodiment, the software compensation described in FIG. 12 may be combined with the hardware compensation described in FIG. 13.

FIGS. 14A and 14B represent a patient trial.

According to a last embodiment, it is possible to consider another form of hardware compensation, the one which consists in recording the totality of the perturbation in a memory of the onboard system and subtracting it directly in real time from the output of the preamplifier.

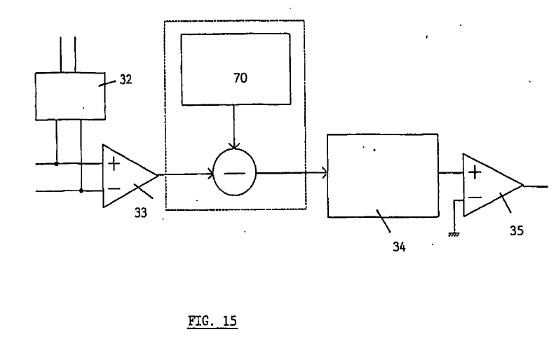

FIG. 15 represents diagrammatically the hardware components intended for the implementation of this solution. This solution is described by the following steps:
before stimulating, short-circuiting via 32 a first time the measurement electrodes 31 and recording the totality of the perturbation at the output of the preamplifier 33.
   an analog digital conversion is performed at the output of the preamplifier,
   the samples are stored in memory
at the moment of the measurement, subtracting directly from the output of the preamplifier 33 and in real time the perturbation of the measured signal.
   The arbitrary function generator 70 may be effected by dispatching the samples previously stored to a digital analog converter.

According to another embodiment, it is possible to propose the use of an instrumentation amplifier having an offset compensation external resistor. The external resistor may be replaced with a digital potentiometer and the μC takes in charge the programmation of the potentiometer so as to cancel the DC component at the output of the preamplifier.

It is also conceivable to envisage the possibility of removing the high-pass filter and replacing it with a summator circuit in order to subtract in real time from the output of the preamplifier the value of the DC component.

b) Stimulation Response without Short-Circuiting of the Electrodes Mode (Mode 1′)

When the acquisition chain does not receive the synchronization signal, it is impossible to short-circuit the measurement electrodes during the stimulation and the acquisition chain must therefore operate in triggering by level mode.

In order to satisfy conditions 3 and 4 established previously for the gain of the whole amplification chain, we shall now consider separately the gain of the preamplifier (G₁) from the gain of the second amplification stage (G₂)

if V_{ARTIFACT}>V_{EMG}, the solution implemented consists in:

performing a preamplification of the signal to be measured as complying with condition (3), i.e.

$${G}_{MAX , 1} = \frac{{V}_{REF}}{{V}_{ARTIFACT}} ,$$

with at the output of the preamplifier “V₀, given by
V₀=G_{MAX,1}·V_{EMG};
filtering this signal so as to preserve only the useful energy band. In this way, condition (4) is again satisfied and the signal may be reamplified in an optimal manner;
amplifying the filtered signal, complying with condition (4), i.e.

$${G}_{MAX , 2} = \frac{{V}_{REF}}{{V}_{0}}$$

if V_{EMG}>V_{ARTIFACT}, then

$${G}_{MAX , 1} = \frac{{V}_{REF}}{{V}_{EMG}} ,$$

this being directly the optimal condition and consequently G_{MAX,2}=1.

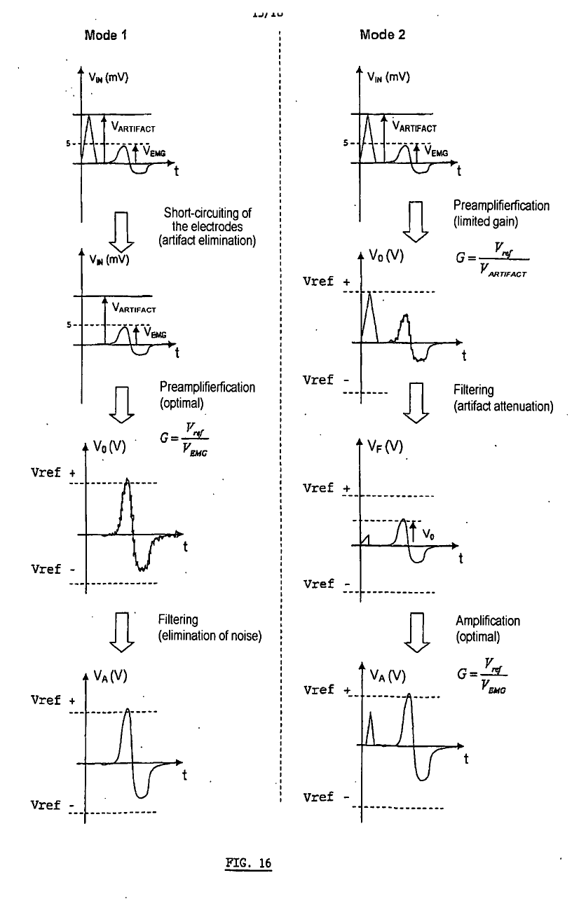

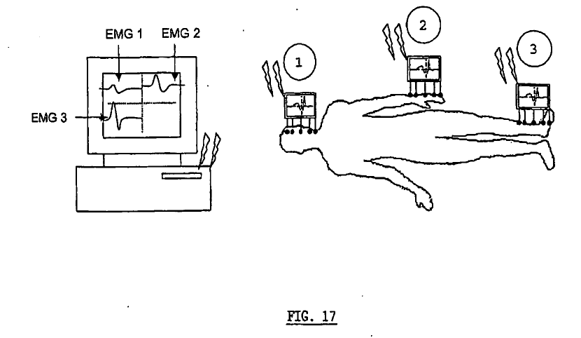

The comparison of the various processing steps in modes 1 and 2 is illustrated in FIG. 16.

In synchronized mode, regardless of the relative amplitude of the EMG with respect to the stimulation artifact, the signal may still be amplified in an optimal manner, that is to say keeping the relative quantization error constant.

In triggering by level mode, a constant relative quantization error is ensured only if the amplitude of the stimulation artifact after filtering drops below the amplitude of the preamplified EMG (which is not always the case in anaesthesia). Indeed, if V_{ARTIFACT (AFTER THE FILTER)}>V_{EMG (AFTER THE FILTER)} then the gain of the second amplifier stage is limited to:

$${G}_{MAX , 2} = \frac{{V}_{REF}}{{V}_{ARTIFACT ( AFTER THE FILTER )}}$$

The total gain of the chain is therefore given by

$${G}_{MAX , TOTAL} = \frac{{V}_{REF}}{{V}_{ARTIFACT}} \cdot \frac{{V}_{REF}}{{V}_{ARTIFACT ( AFTER THE FILTER )}} < \frac{{V}_{REF}}{{V}_{EMG}}$$

The short-circuiting of the electrodes therefore presents a double advantage:
the maximum possible gain may be set at the level of the preamplifier

$${G}_{MAX , 1} = \frac{{V}_{REF}}{{V}_{EMG}} ,$$
this makes possible to increase the signal-to-noise ratio
the total gain of the chain can be dimensioned in an optimal manner:

$$by taking {G}_{MAX , 2} = 1 \to {G}_{MAX , TOTAL} = \frac{{V}_{REF}}{{V}_{EMG}}$$
this making it possible to keep the relative quantization error constant regardless of the amplitude of the EMG in relation to the one of the stimulation artifact.
   Safety Aspects

The system according to the invention is designed to carry out regularly or on demand the measurement of impedance at the levels of the acquisition electrodes.

Specifically, in anaesthesia, it is imperative to distinguish between the decrease in the amplitude of the EMG due to the effects of the curare and the decrease due to the detaching of the measurement electrodes.

The system comprises protection resistors for limiting the default current in case the supply voltage would be applied accidentally to the measurement electrodes.

1.4 Modes of Operation of the Appliance

It follows from the above descriptions that the system is provided for operating in four different modes and its architecture is adapted for keeping the quantization error constant in the first three modes.
stimulation-response:
   with short-circuiting of the measurement electrodes
   without short-circuiting of the measurement electrodes
inverted
acquisition only
stimulation only
   1.5 Complementary Functionalities

“Wireless” Transmitter/Receiver

The use of “wireless” technology affords:
the possibility of remote measurement taking (hence wireless) and decentralized management of the appliance;
the possibility of networking for polytopic measurement (see FIG. 17).

The appliance is designed to be able to work as a “slave” of a central computer via a wireless connection. The central computer is moreover able to establish a communication with several EMG stimulation-response systems and to interrogate them in turn (FIG. 19).

Saving of the EMGs

The EMGs are recorded on the memory card of the PDA.

1.6 Advantages of the Invention

To summarize, a certain number of advantageous characteristics of the appliance according to the present invention make it possible to distinguish this appliance from the known prior art.

Masking of the Stimulation Artifact

the acquisition chain is not disrupted by the stimulation artifact;
possibility of amplifying under optimal conditions regardless of the amplitude of the EMG;
improvement in the signal-to-noise ratio.
   Master or slave operation—“Master/Slave”
the system can work in a completely autonomous manner (master PDA);
the system can work as slave of a central computer so as to perform measurements on demand (slave PDA).
   Wireless Link with the PDA
The system uses for example a “Bluetooth” transmitter/receiver integrated into the PDA to communicate with another computer.
   Safety
The system of the invention consumes a minimum; entirely battery based, it avoids problems related to galvanic isolation; this system is particularly well suited to a polytopic measurement (no common earth for the various sensors);
the system furthermore comprises protection resistors so as to adhere to medical standards which prescribe that the default current must be limited to 50 μA.
   Multi-Topical
Since it deals with an electrical measurement, the appliance of the invention is suitable whenever needles or electrodes can be placed. It is suitable for the hand but also for any other site.
   Reliability
The system performs a test of detachment of the stimulation and acquisition electrodes before each measurement campaign and warns when the stimulation or acquisition electrodes are poorly positioned, for example when an electrode is poorly attached.
   Flexibility

Programmability of the elementary stimuli, of their sequencing or of their repetition over time:
the stimulator can provide prerecorded pulse trains;
the stimulator can provide waveforms drawn by the user.
   Reversibility of the Appliance
stimulation-response mode;
inverted mode.
   Automatic Gain Control
The system automatically adjusts the gain of the amplifiers to preserve the resolution when the amplitude of the EMGs decreases.
   Fixed Resolution
The invention cunningly utilizes the magnitude of the signal, by automatically adapting the amplification of the measurement chain to exploit the maximum of the resolution of the ADC of the system.
   Schedule
The user can program various modes of stimulation and the moment at which he wishes to deliver them, this being useful in an operating theatre for example (curarization phase, operating phase, decurarization phase).
   Complementary Analysis in Post-Processing

The user can obtain complementary information in post-processing:
peak-to-peak amplitude;
rectified EMG area;
spectral analysis, etc.;
   as well as the ratio of certain of these measurements.
   2. Areas of Application
   2.1 Point of View of the Person Administering the Drugs (Curare) and of Neurophysiologists

The appliance according to the invention is first and foremost useful for assessing the effect of new molecules which appear on the market and whose effects on various muscles must be estimated on various sites.

Indeed, on the one hand, the time constant and the inertia of the effects of the curarizing agents depend on the type of drug administered to the patient and, on the other hand, the rate of paralysis is not uniform in all parts of the body.

The appliance described is also useful during neurophysiological examination for the evaluation of muscle tone. One is often required to assess the muscular toneness of a given muscle or the relationship of paralysis or of recovery between two muscles. Specifically, when injecting curare, the paralysis of the patient begins at the central level and terminates in the distal muscles. Likewise, muscles like the diaphragm are paralyzed before the muscles situated at the extremities, such as the thumb adductor. The decurarization process takes place in the same order.

For example, if only the foot is accessible, one would wish to be able to assess its muscular toneness and moreover ascertain its relationship with the muscular toneness of the larynx and the muscular toneness of the diaphragm so as to know when it is possible to intubate or extubate a patient.

2.2 Point of View of Anesthetists

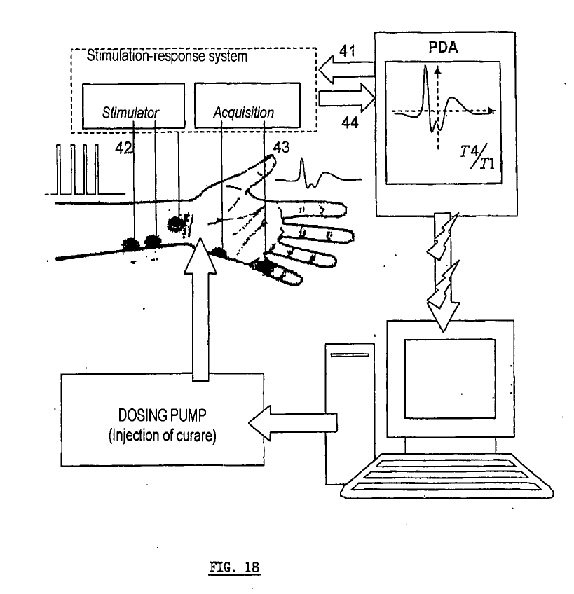

From the point of view of anaesthetists, the appliance according to the present invention is useful on two accounts:
firstly in an operating theatre for assessing the degree of neuromuscular blockade when curarizing a patient in open or closed loop (administration by single bolus, repeated bolus, continuous perfusion);
thereafter for evaluating the recovery of the neuromuscular function of a patient after a surgical intervention, the internal muscle groups being those which are involved in respiration and in protecting the upper airways.
   Closed Loop Acquisition in Operating Theatre (FIG. 18)

The PDA is capable of communicating with a central computer via a WIRELESS connection or a wire connection with galvanic isolation. The appliance may therefore work as slave of the master computer and be integrated into a closed regulating loop.

After having performed the measurement of the EMG, the PDA dispatches information (totality of the curve or preprocessed response) to the central computer which drives the pumps for injecting the curare.

The PDA operates as slave of a workstation. The central computer periodically interrogates the stimulation-response system so as to supervise the degree of neuromuscular blockade of the patient during the surgical intervention. The regulating loop is of closed type. The central computer also supervises the injection of the curare pumps.

Advantage Afforded by the Association of Stimulator and Acquisition System (FIG. 19)

In electromyography (EMG), the evaluation of the degree of neuromuscular blockade is done by evaluating the response of the muscle (potential evoked) to a “supra-maximal” electrical stimulation of a peripheral motor nerve. If the reaction of a single muscle fiber is of the “all or nothing” type, the reaction of the entire muscle depends on the number of active fibers.

A stimulation of sufficient intensity will cause the reaction of the totality of the muscle fibers and the response obtained will be a maximum in amplitude.

FIG. 19 shows the EMG obtained when the intensity of the stimulation current source is progressively increased.

The amplitude of the response signal increases with the intensity of the current pulses until saturation is reached. This saturation indicates that the totality of the muscle fibers are in fact excited and the response reaches a maximum amplitude.

Seeing that the administration of curare decreases the number of active fibers, it is possible to relate the weakening of the maximum response with the state of relaxation of the muscle.

For this technique to be efficacious, it is vital that the totality of active fibers are excited by the stimulation.

The intensity of this stimulation will therefore be 20 to 25% greater than the one for which a maximum response is obtained, hence the term “supra-maximal”.

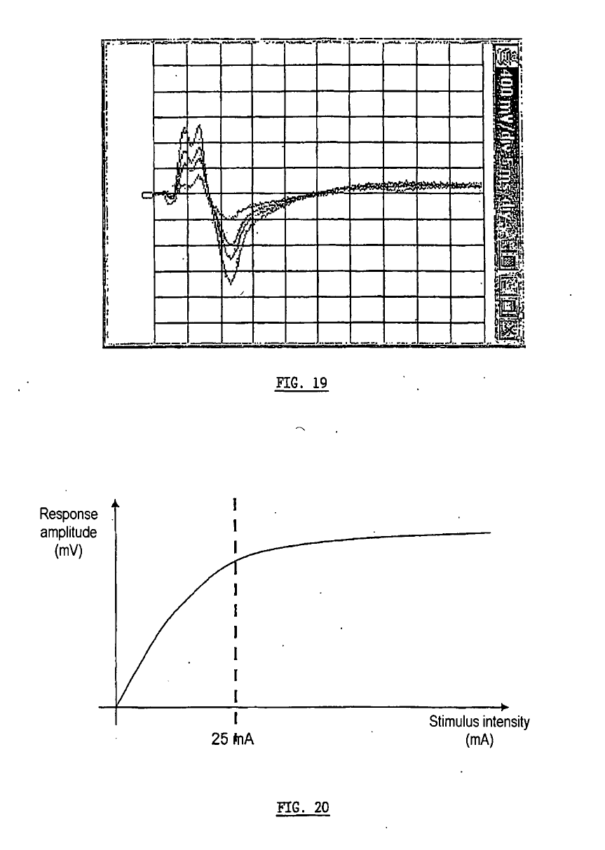

FIG. 20 shows the amplitude of the EMG signal as a function of the intensity of the electrical pulses.

Experience shows that the threshold, characterized by the saturation of the amplitude, depends strongly from one muscle to another and even from one patient to another.

Certain appliances such as the TOF-Watch used currently (in accelerometry) are programmed by default to 50 mA so as to be sure of being beyond the saturation threshold and that the muscle is correctly excited.

Furnished with a measurement of the EMG, the microcontroller can determine very accurately the intensity of the electrical pulses leading to a supra-maximal excitation.

One of the numerous advantages in using EMGs for the evaluation of the rate of muscle relaxation is the much finer detection of the saturation threshold allowing to decrease the intensity of the electrical pulses and, consequently, to thereby decrease post-operative pain.

1-30. (canceled)

31. An appliance device for the direct measurement, display, processing and transmission, remotely, of electromyographic signals (EMG) comprising:
an electrical stimulator comprising electrodes for the excitation of a peripheral motor nerve;
a pair of electrodes, for the acquisition of the EMG response at the level of the muscle associated with this peripheral nerve;
an acquisition chain driven by a microcontroller, presenting conditioning elements of the input signal, comprising at least one differential preamplifier, a bandpass filter and an analog/digital converter (ADC), said acquisition chain being linked, via a standardized interface, to a computer comprising storage and display functionalities of the EMG signals acquired as well as an executable program for effecting the interface with the user and utilizing the data stored;
wherein the acquisition chain allows the automatic adjustment of the amplification gain of the EMG signal, via the microcontroller, in such a way that the EMG signal covers the largest possible part of the input voltage span of the ADC, with conservation of resolution, when the amplitude of the EMG signal decreases.

32. The appliance device according to claim 31, intended for anaesthesia monitoring applications, wherein the elimination or of attenuation of a stimulation artifact present in the EMG signal is performed.

33. The appliance device according to claim 32, wherein said elimination and/or attenuation of the artifact is performed through a relay driven by the microcontroller, for short-circuiting the acquisition electrodes for the duration of the appearance of the stimulation artifact.

34. The appliance device according to claim 33, wherein the short-circuiting of the electrodes is performed in such a way as to make it possible to synchronize the start of the acquisition with the end of the stimulation.

35. The appliance device according to claim 34, wherein the duration of short-circuiting of the electrodes is programmable and is preferably between 1 and 10 000 μs, preferably between 1 and 1000 μs, and in that the duration of acquisition of the signal is programmable and is preferably between 1 and 60 000 ms, preferably between 1 and 10 000 ms.

36. The appliance device according to claim 33, wherein the elimination the artifact of the offset appearing during a short-circuiting of the measurement electrodes is performed.

37. The appliance device according to claim 36, wherein the elimination of the artifact of the offset is performed by a hardware compensation.

38. The appliance device according to claim 37, wherein the hardware compensation allowing the elimination of the artifact of the offset comprises an adder circuit and an offset generator, the high-pass filter of the bandpass filter being omitted.

39. The appliance device according to claim 37, wherein the hardware compensation allowing the elimination of the artifact of the offset comprises an amplifier with offset compensation external resistance, the high-pass filter of the bandpass filter being omitted.

40. The appliance device according to claim 37, wherein the hardware compensation allowing the elimination of the artifact of the offset comprises a Sample & Hold sampler associated with an analog multiplexer.

41. The appliance device according to claim 37, wherein the hardware compensation allowing the elimination of the artifact of the offset comprises a recordation allowing the recording of the perturbation in RAM with the presence of an adder circuit.

42. The appliance device according to claim 36, wherein the elimination of the offset is performed by a software compensation.

43. The appliance device according to claim 31, wherein the electrodes are surface electrodes, needle electrodes and active electrodes.

44. The appliance device according to claim 31, wherein the acquisition comprises a second amplifier for the processing of the signal after preamplification and filtering, said second amplifier also comprising means of automatic adjustment of the gain, via the microcontroller.

45. The appliance device according to claim 31, which further comprises protection resistors for limiting the default current.

46. The appliance device according to claim 31, which is configurable so as to allow the acquisition and the transmission, remotely, of data in real time.

47. The appliance device according to claim 31, which is configured to perform automatically or on demand a measurement of impedance at the level of the stimulation electrodes and of the acquisition electrodes.

48. The appliance device according to claim 31, wherein the stimulator is configured to work with a series of sequences of reparametrizable rectangular pulses being prerecorded in memory, preferably of ST (Single Twitch), TOF (Train Of Four), TS (Tetanic Stimulation) or DBS (Double Burst Stimulation) type.

49. The appliance device according to claim 48, wherein the amplitude of the pulses is between 0 and 150 mA in 5 kΩ, their width between 0 and 1000 μs and the period separating two successive sequences between 1 and 60 000 ms.

50. The appliance device according to claim 49, wherein the pulses are sinusoidal, triangular, trapezoidal, rectangular or arbitrary.

51. The appliance device according to claim 50, wherein the stimulator can be configured to work in monopulse, multipulse or continuous mode.

52. Method for the direct measurement, display, processing and transmission, remotely, of electromyographic signals (EMG) by means of an appliance device according to claim 31, wherein an automatic adjustment of the amplification gain of the EMG signal is performed, via the microcontroller, in such a way that the EMG signal covers the largest possible part of the input voltage span of the ADC, with conservation of resolution, when the amplitude of the EMG signal decreases.

53. The method according to claim 52, wherein there is performed an elimination or an attenuation of the stimulation artifact present in the EMG signal by short-circuiting the acquisition electrodes for the duration of the appearance of the stimulation artifact.

54. The method according to claim 53, wherein there is performed a software or hardware compensation of the EMG signals to eliminate the artifact of the offset.

55. The method according to claim 54, wherein, for a software compensation, the following steps are performed:
short-circuiting the measurement electrodes without electro-stimulating and recording the perturbation at the output of the measurement chain (V_{PERTURBATION}),
performing the measurement of the EMG evoked, the measured signal then being the superposition of the EMG evoked and of the perturbation related to the high-pass filter, i.e.:
(V_{MEASURE}=V_{EMG}+V_{PERTURBATION}),
subtracting the perturbation signal from the measured signal and displaying the result, i.e.:
(V_{DISPLAY}=V_{MEASURE}−V_{PERTURBATION}).

56. The method according to claim 54, wherein, to perform a hardware compensation, the following steps are performed:
before stimulating, sampling the output of the preamplifier and storing the sample by means of a sampler,
connecting the output of the sampler to the input of the bandpass filter by means of an analog multiplexer,
short-circuiting the measurement electrodes through the element,
de-short-circuiting the measurement electrodes, and
reconnecting the input of the bandpass filter to the output of the preamplifier.

57. The method according to claim 54, characterized in that, for a hardware compensation, the following steps are performed:
before stimulating, short-circuiting via a short-circuiting a first time the measurement electrodes and recording the totality of the perturbation at the output of the preamplifier,
performing an analog digital conversion at the output of the preamplifier,
storing the samples in memory,
at the moment of the measurement, subtracting directly from the output of the preamplifier and in real time the perturbation of the measured signal.
